(19) United States
(12) Patent Application Publication
(10) Pub. No.: US 2013/0267798 A1
(43) Pub. Date: Oct. 10, 2013

(54) NONINVASIVE MEASUREMENT OF ANALYTE CONCENTRATION USING A FIBERLESS TRANSFLECTANCE PROBE

(71) Applicants: Hannu HARJUNMAA, Holden, MA (US); Stevan Kun, Worcester, MA (US)

(72) Inventors: Hannu HARJUNMAA, Holden, MA (US); Stevan Kun, Worcester, MA (US)

(21) Appl. No.: 13/441,447

(22) Filed: Apr. 6, 2012

(51) Int. Cl. A61B 5/1455; A61B 5/00

(52) U.S. Cl. 600/316; 600/310

(57) ABSTRACT

A method and apparatus for noninvasively measuring the concentration of a target analyte in a sample matrix using a fiberless transflectance probe is described. It includes directing a beam of electromagnetic radiation, consisting of at least two components of different wavelengths, to the sample matrix and conducting the backscattered radiation to a detector which outputs a signal indicative of the differential absorption of the two wavelengths in the sample matrix. The transflectance probe comprises a tapered tubular housing having an inner reflective surface, an optical rod having an outer reflective surface, and a detection window which serves as an interface between the probe and the surface of the sample matrix. The method and apparatus described are particularly useful in measuring the concentration of glucose in tissue containing blood.

The present disclosure generally relates to the field of biomedical testing. More specifically, the present disclosure relates to methods and apparatus for noninvasive measurement of concentration of analytes in body tissues.

### BACKGROUND

Noninvasive diagnosis and measurement of blood glucose concentration has attracted tremendous attention in the past two decades because of the emergence of diabetes as an epidemic, particularly when associated with an increased overall obesity of the population. Noninvasive measurement of glucose offers the potential for increased frequency of testing, and thus, enable tighter control of blood glucose concentrations through concomitant adjustment of insulin doses. Noninvasive detection techniques also offer the potential for a portable, closed-loop system for monitoring and regulating insulin dosage. These prospective advantages have led to considerable interest in the commercialization of noninvasive glucose monitoring devices.

Currently, all available portable end-user devices for measuring blood glucose require puncturing the fingertip to obtain a blood sample. The blood sample is then placed on a test strip that indicates the glucose concentration. These devices are very compact and reasonably accurate, but puncturing the fingertip to obtain a blood sample is inconvenient, painful, and poses a risk of infection. Noninvasive devices for measuring blood glucose are not commercially available at present.

A number of attempts have been made to measure blood glucose concentration noninvasively by measuring tissue absorption of light radiation in the near infrared energy spectrum-approximately 650 nm to 2700 nm. U.S. Pat. No. 5,099,123 to Harjunmaa et al., which is incorporated herein in its entirety by reference, discloses a balanced differential (or Optical Bridge™) method for measurement of analyte concentration in turbid matrices, i.e. body fluids and tissue. The method utilizes two wavelengths—a principle wavelength which is highly absorbed in the target analyte, and a reference wavelength, selected using a balancing process, which is not (or much less) absorbed in the target analyte. The two wavelengths are selected to have substantially identical extinction coefficients in the background matrix. When a radiation beam comprising the two wavelengths in alternate succession is applied to the sample tissue matrix, an alternating signal synchronous with the wavelength alternation is registered in a signal detector measuring the radiation transmitted or backscattered by the matrix. The amplitude of the alternating signal is proportional to the concentration of the target analyte in the sample matrix. During the measurement, the Optical Bridge balancing process is used to vary the two alternating wavelengths and their relative intensities such that in the absence of analyte, the detector signal is essentially zero. That is, the Optical Bridge uses the two near infra-red wavelengths to “null out” the background absorption so that the analyte concentration becomes much more visible.

Subsequently, in U.S. Pat. No. 5,178,142, which is incorporated herein by reference, Harjunmaa et al. disclosed a method of changing the extracellular to intracellular fluid ratio of the tissue matrix by varying the mechanical pressure on the tissue, and zeroing the transmitted/reflected signal (balancing) when there is a minimum level of analyte present in the sample.

In U.S. Pat. No. 7,003,337, which is incorporated herein by reference, Harjunmaa et al. disclosed continuous estimation of the amount of fluid containing the target analyte within the sample using another radiation (such as green light which is absorbed by hemoglobin), and combining the output of the sample detector with the fluid volume estimate to calculate the analyte concentration. Further, in U.S. application Ser. No. 11/526,564, which is also incorporated herein by reference, Harjunmaa et al. disclosed a method of producing a radiation beam using three fixed-wavelength laser diodes instead of tuning the laser wavelengths during use.

Other related patents include U.S. Pat. Nos. 5,112,124; 5,137,023; 5,183,042; 5,277,181 and 5,372,135, each of which is incorporated by reference herein in its entirety.

### SUMMARY

The present disclosure describes a method and apparatus for noninvasively measuring the concentration of a target analyte in a sample using a fiberless transflectance probe. A first aspect of the present disclosure is an illustrative apparatus for noninvasively interrogating a target region for measuring an amount of a target analyte, wherein the apparatus comprises a source for generating a combined beam of electromagnetic radiation including at least two repetitive periods of radiation having different wavelengths, at least two of the wavelengths having different absorption coefficients for the target analyte. The apparatus further comprises a detector arranged to detect a portion of the radiation backscattered by the target region, the detector generating an output signal proportional to the detected intensity of the combined beam at each of the two repetitive periods of radiation, and a fiberless transflectance probe for directing the beam of electromagnetic radiation to the target region and conducting the backscattered light to the detector, wherein the fiberless transflectance probe comprises a tapered tubular housing with an inner reflective surface, a cylindrical optical rod with an outer reflective surface and a detection window through which the radiation beam is transmitted to the target region.

Another aspect of the present disclosure is an illustrative transflectance probe for measuring a property of a sample, which includes a detection window through which the sample is irradiated, an optical rod with an outer reflective surface positioned perpendicular to the detection window, a tapered tubular housing with an inner reflective surface positioned around the optical rod, at least one light source for irradiating the sample, and a detector positioned at the proximal end of the optical rod for detecting the light backscattered by the sample.

Yet another aspect of the present disclosure is an illustrative method of noninvasively interrogating a target region for measuring an amount of a target analyte, comprising the steps of providing a fiberless transflectance probe comprising a tapered tubular housing with an inner reflective surface, a detection window and an optical rod with an outer reflective surface positioned perpendicular to the optical rod. The method further includes providing at least two light sources operating at two different wavelengths for generating a radiation beam consisting of at least two time multiplexed components, transmitting the radiation beam to the target region by reflecting on the inner surface of the tubular housing and the outer surface of the optical rod, conducting the backscattered beam from the target region to the detector by reflecting on the inner surface of the optical rod, and providing a detector that detects the backscattered beam and produces an output signal indicative of the differential absorption of the two wavelengths by the target region.

It is to be understood that both the foregoing general description and the following detailed description are exemplary and explanatory only and are not restrictive of the invention, as claimed.

### BRIEF DESCRIPTION OF DRAWINGS

The accompanying drawings, which are incorporated in and constitute a part of this specification, illustrate embodiments of the invention and together with the description, serve to explain the principles of the various aspects of the invention.

FIG. 1 is a schematic diagram of an analyte testing device, in accordance with an embodiment of the present disclosure;

FIGS. 2A and 2B illustrate the operation of the Optical Bridge, in accordance with an embodiment of the present disclosure;

FIG. 3A is a schematic diagram of an illustrative fiberless transflectance probe embodiment;

FIG. 3B is a schematic diagram of the distal end of the fiberless transflectance probe embodiment illustrated in FIG. 3A; and

FIG. 4 illustrates the distribution of the incident radiation beam on a measurement site, in accordance with an embodiment of the present disclosure.

### DETAILED DESCRIPTION

Reference will now be made in detail to embodiments consistent with the present disclosure, examples of which are illustrated in the accompanying drawings. Wherever possible, the same reference numbers will be used throughout the drawings to refer to the same or like parts.

In an exemplary embodiment, an optical system comprising a fiberless transflectance probe is used to measure the concentration of a target analyte in a fluid within a sample matrix. The analyte concentration is measured and analyzed using a portable device developed using Optical Bridge™ technology. In accordance with an embodiment of the present disclosure and the Optical Bridge™ technology, noninvasive optical measurements of the analyte concentration are performed using a beam of electromagnetic radiation which alternates at a particular frequency between a “principal” wavelength (λ₀), a “reference” wavelength (λ₁) and an auxiliary wavelength λ₂. λ₀ is selected to achieve high analyte absorption, and λ₁ is selected to have minimal analyte absorption. During the Optical Bridge™ balancing step, λ₁ is adjusted to have the same absorption in blood-less tissue as λ₀. The auxiliary wavelength λ₂ is selected to have high absorption in a component of the fluid, and is used to provide an estimate of the fluid content of the sample matrix. In an exemplary embodiment of the present disclosure, the fiberless transflectance probe is used to measure the concentration of glucose (i.e. the target analyte) in blood (i.e. the fluid). In such as embodiment, λ₀ is selected to be about 1620 nm and λ₁ is selected to be about 1380 nm, which are in the near infrared energy spectrum. The auxiliary wavelength λ₂ is selected to be about 525 nm, which is an isosbestic wavelength for hemoglobin, and provides an excellent sensitivity to blood. In one such embodiment, the three wavelengths, λ₀, λ₁, and λ₂, are 1620+/−20 nm, 1380+/−20 nm, and 525+/−20 nm, respectively. The beam of electromagnetic radiation consists of time multiplexed component of the three different wavelengths (λ₀, λ₁ and λ₂) alternating at a frequency of 100 Hz. In another embodiment, some or all wavelengths are on at all times, i.e., they are not alternating. In certain embodiments, the separation of the signal into its wavelength components is performed by the detector or processor.

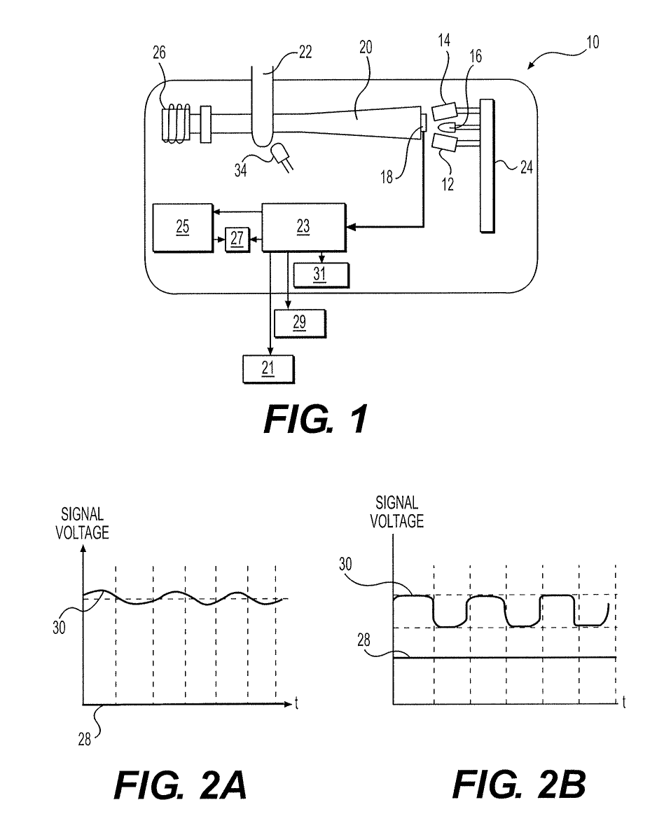

FIG. 1 shows a conceptual diagram of an analyte testing device 10 which utilizes the Optical Bridge™ technology for noninvasively measuring the concentration of a target analyte (e.g. glucose) in a fluid (e.g. blood) within a sample matrix (e.g. a tissue matrix). The analyte testing device 10 comprises at least two laser diodes 12 and 14 operating at wavelengths λ₀ and λ₁ respectively, a signal detector 18, and an optical transflectance probe 20 which interfaces the laser diodes with a measurement site 22. In one embodiment, the analyte testing device 10 further comprises at least one LED 16 operating at a wavelength λ₂. The beam through the optical probe 20 alternates between λ₀, λ₁ and λ₂ at a preselected frequency. The wavelength alternation is driven by the laser controller module 24. The measurement site 22 is chosen such that it is: 1) easily accessible, 2) well perfused with the fluid containing the target analyte; 3) small enough to fit in a sample port of a portable instrument, 4) can be easily compressed/ uncompressed. In one embodiment of the present disclosure, a subject's earlobe is used as the measurement site 22. In another embodiment, the subject's finger is used as the measurement site 22.

In an exemplary embodiment, the extracellular-to-intracellular fluid ratio of the measurement site 22 is changed during the measurement by exerting varying mechanical pressure on the measurement site. In such an embodiment, the amount of fluid in the measurement site 22 is modulated by means of a linear actuator 26, as illustrated in FIG. 1. The linear actuator compresses the measurement site 22 with a pressure sufficient to displace fluid (with the target analyte) from the measurement site 22. In one such embodiment, the linear actuator compresses the measurement site 22 with a pressure three times systolic blood pressure. As the compressive force is released, the displaced fluid returns to the measurement site. In one embodiment, linear actuator 26 compresses measurement site 22 against optical probe 20. In another embodiment, linear actuator 26 compresses optical probe 20 against measurement site 22.

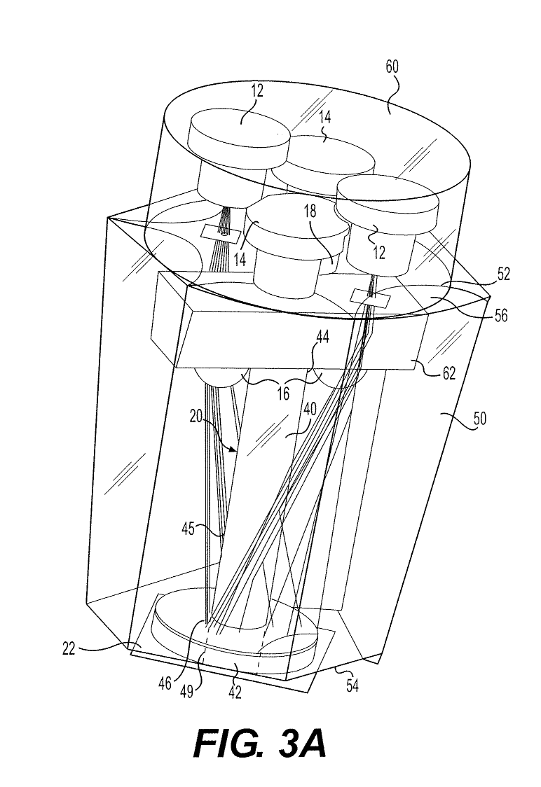

The Optical Bridge™ technology exploits the principle that compressed tissue has a relatively lower proportion of fluid with the target analyte than uncompressed tissue, although some residual amount of analyte remains in the measurement site 22 during the compression. In another embodiment, the extracellular-to-intracellular fluid ratio is allowed to change as a result of natural pulsation due to heartbeat, and the measurement cycle is synchronized with such pulsation. When the extracellular fluid volume in the measurement site is reduced either due to mechanical compression or natural pulsation, the optical path of the radiation beam contains minimal fluid and the target analyte. The Optical Bridge™ balancing is performed at this position at the beginning of each measurement to achieve the maximum background rejection. The balancing is performed by adjusting the light intensities at the two wavelengths λ₀, λ₁, and also by modifying the reference wavelength λ₁. The variations in the background matrix structure are compensated for in the balancing process. As indicated in FIG. 2A, the light intensities and the wavelength λ₁ are adjusted such that the baseline absorption (indicated by the Optical Bridge Signal 28) is essentially zero when there is minimal fluid and analyte in the optical path, and the differential absorption of the wavelengths λ₀ and λ₁ (indicated by the variation in Detector Output Voltage 30) is minimum. The Optical Bridge Signal 28 is in effect the rectified Detector Output Voltage 30.

In an exemplary embodiment which utilizes the compression mechanism, the pressure on the measurement site 22 is relaxed after the Optical Bridge is balanced, allowing fluid to return to the site. The attenuation of the two wavelengths λ₀ and λ₁ is different at the uncompressed position, as indicated by the larger variation in the Detector Output Voltage 30 in FIG. 2B. At the uncompressed position, the Optical Bridge Signal 28 is higher (i.e. there is more background absorption in the measurement site 22), as indicated in FIG. 2B. Variations in the Detector Output Voltage 30 is proportional to the changes in the amount of target analyte (e.g. glucose) in the fluid. In order to accurately calculate the concentration of the analyte in the fluid, the variations of the amount of fluid in the measurement site must also be measured. The wavelength λ₂ which is highly absorbed by a component of the fluid, and follows the same optical path as wavelengths λ₀ and λ₁, is used to compensate for the changes in fluid volume in the measurement site. Features extracted from the detected λ₂ signal are processed to produce an estimate of the fluid volume, which is then combined with the detected λ₀ and λ₁ signal output to produce an estimate of the concentration of analyte in the blood.

In one embodiment, an auxiliary radiation source 34, as illustrated in FIG. 1, is used to detect pulse and to synchronize the measurement with the inrush of blood into the measurement site 22. In one embodiment, the auxiliary radiation source 34 is a LED operating at 525 nm (an isosbestic wavelength for hemoglobin). The auxiliary radiation source 34 is directed at a portion of the sample matrix that maintains good circulation at all times. For example, the radiation source 34 may be directed at a portion of the sample matrix, outside the measurement site 22, which is not compressed by the linear actuator 26. The radiation source 34 generates a pulse detection beam which is scattered by the tissue, and a fraction of the original beam is detected by the signal detector 18. The auxiliary radiation source 34 is operated prior to the measurement step to synchronize the start of the measurement process with a variation of the blood pressure.

In one exemplary embodiment, optical probe 20 is configured for transflectance measurements, wherein the radiation beam is inserted into the measurement site 22 and the backscattered beam is detected by the signal detector 18. The detector then generates a signal indicative of the differential absorption of the target analyte. An important consideration for such an embodiment is that the light reflected from the surface of the measurement site 22 should not reach the detector as it would overwhelm the backscattered light.

In one such embodiment, transflectance measurement is performed using a bifurcated bundle of optical fibers, a first portion of which is adapted to receive light from the laser diodes operating at wavelengths λ₀ and λ₁, and a second portion of which is adapted to conduct the backscattered light to the signal detector. The fiber bundle passes through the optical probe 20, and the common end of the fiber bundle is pressed against the measurement site 22 for the transflectance measurements.

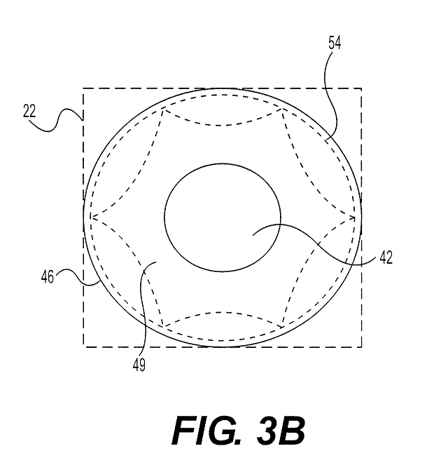

In another embodiment, transflectance measurement is performed using a fiberless transflectance probe 20, as illustrated in FIG. 3A. The transflectance probe 20 interfaces laser diodes 12, 14, at least one LED 16 and the sample detector 18 with the measurement site 22. Transflectance probe 20 comprises a cylindrical optical rod 40 having a polished outer surface 45. In one embodiment, the optical rod 40 is made of fused quartz and the outer surface 45 is coated with aluminum to increase the reflectivity of the surface. In another embodiment, the optical rod 40 is a glass rod with aluminum coating on the outer surface 45. Optical rod 40 is positioned perpendicular to a round detection window 46. The distal end 42 of optical rod 40 is inserted into a circular opening 44 in the detection window 46, such that the distalmost end of the optical rod is axially aligned with the distal surface 49 of the detection window, and is in direct contact with the surface of the measurement site 22. In order to limit interaction between the incident light and the backscattered light, the optical rod 40 is coated with aluminum throughout its length, including the distal end 42 which is inserted into the detection window 46. Additionally, the optical rod 40 and the detection window 46 are tightly coupled to ensure that a substantial portion of the radiation backscattered from the measurement site 22 enters the optical rod 40.

The electromagnetic radiation beam is transmitted to the measurement site 22 through the detection window 46 during a measurement. Thus, the detection window 46 acts as an interface between the sample matrix and device hardware. The detection window 46 is also used to apply mechanical pressure on the measurement site 22 during a compression/decompression procedure, as described earlier. In one embodiment consistent with the present disclosure, the detection window 46 is comprised of glass or quartz. In another embodiment, the detection window 46 is comprised of a thermoplastic polymer that has high transmittance in the wavelength range consisting of λ₀, λ₁, and λ₂, has low moisture absorbility and is suitable for injection molding. Example of such thermoplastic polymers include, but is not limited to, cyclic polyolefins (COP), polymethylmethacrylate (PMMA), and polystyrene (PS).

The optical rod 40 is further surrounded by a tapered tubular housing 50 having an inner reflective surface. In one embodiment, the inner surface 52 is aluminized to increase the reflectivity of the surface. The distal end 54 of the tapered tubular housing 50 is coupled with the detection window 46, as shown in FIG. 3B. In one embodiment consistent with the present disclosure, the tapered tubular housing 50 is made of quartz or glass. In another embodiment, the tapered tubular housing 50 is made of a thermoplastic polymer using injection molding, and the inner surface 52 is coated with aluminum to increase the reflectivity of the surface. In yet another embodiment, the detection window 46 and the tapered tubular housing 50 are injected molded together using the same thermoplastic polymer.

The tapered tubular housing 50 also facilitates shaping of the radiation beam emitted by the laser diodes and the LEDs. The shape of the inner surface 52 and taper angle of the tubular housing guides the distribution of the emitted beam on the measurement site 22. In one preferred embodiment consistent with the present disclosure, the tubular housing 50 is configured as a truncated conical shell having a cone angle (angle between the longitudinal angle and wall) of 7.5°. In another embodiment, the inner surface of tapered tubular housing 50 is faceted in order to distribute the incident light evenly on the measurement site 22. The number of facets in the tubular housing corresponds to the number of laser diodes and LEDs used in the optical probe 20. In one embodiment, the optical probe 20 includes four laser diodes (two each for the wavelengths λ₀ and λ₁), and two LEDs operating at wavelength λ₂. In such an embodiment, the inner surface 52 of the tapered tubular housing 50 has a faceted hexagonal shape, as shown in FIGS. 3A and 3B. The facets on the inner surface 52 are in the form of a convex cylinder, and the radius of curvature of each facet is optimized for the corresponding light source to provide an uniform distribution of light from the different sources on the measurement site 22. In an exemplary embodiment of the present disclosure, the fiberless transflectance probe 20 is used in a optical detection system to measure the concentration of glucose in blood. In such as embodiment, λ₀ is selected to be 1620 nm and λ₁ is selected to be 1380 nm, and the radii of curvature of the cylindrical facets associated with the lasers operating at λ₀ and λ₁ are 7.2 mm and 6.1 mm, respectively. Additionally, the distance of the laser diodes 12, 14 from the central longitudinal axis of the tubular housing guides the distribution of the emitted beam on the measurement site 22. In an embodiment for measuring the concentration of glucose in blood, as discussed above, the distance of the laser diodes from the central axis is 5.3 mm.

The laser diodes 12, 14 are mounted on a heat sink 60 at the proximal end 56 of tapered tubular housing 50 for temperature stability. In one embodiment, the LEDs 16 are also mounted on the heat sink adjacent to the laser diodes. In another embodiment, the LEDs are mounted on a positioning plate 62 below the heat sink 60, as shown in FIG. 3A, to maintain a stabilized operating condition for the laser diodes.

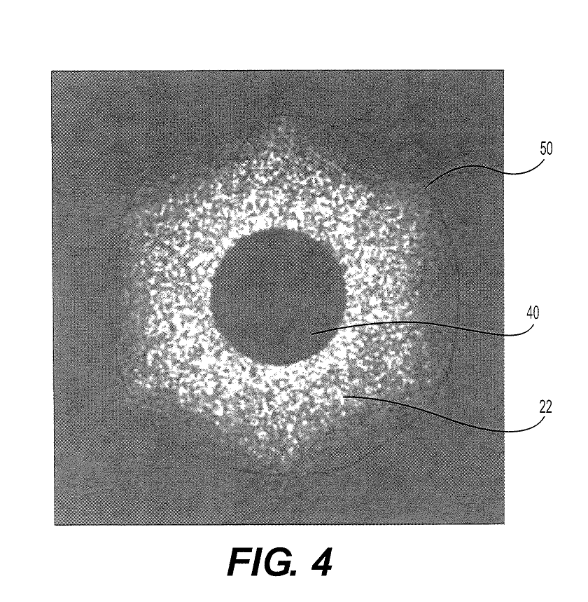

The radiation beam comprising the wavelengths λ₀, λ₁, and λ₂, is transmitted to the measurement site 22 by reflecting on the outer surface 45 of the optical rod 40 and the inner surface 52 of the tapered tubular housing 50. FIG. 4 shows the distribution of light from four laser diodes on the measurement site 22. As shown in the figure, the light from the multiple sources is distributed angularly uniformly on the measurement site, with the area surrounding the optical rod 40 receiving more radiation than the area around the edge of the tubular housing 50. Some of the light incident on the measurement site 22 is backscattered by the sample, and a fraction of the backscattered light reaches the interior of the optical rod 40 and is conducted to the signal detector 18 by reflecting on the inner surface of the optical rod. The sample detector 18 (not shown in FIG. 3A) is positioned at the proximal end 44 of the optical rod 40. When the backscattered light reaches detector 18, an alternating signal is generated which is proportional to the differential absorption of wavelengths λ₀ and λ₁ by the fluid in the sample matrix. The concentration of the target analyte in the fluid is then calculated from the output signal using a signal processing algorithm.

In one exemplary embodiment consistent with the present disclosure and the Optical Bridge™ technology, the analyte testing device 10 is a handheld unit. Referring again to FIG. 1, the handheld unit comprises a screen 27 for graphic display of the measurement results, and the on-board electronics consist of a processor 23 for operating the device and calculating the target analyte concentration, and a control module 24 for driving the laser diodes 12, 14 and LEDs 16. The handheld unit may be powered from an external power supply, rechargeable batteries, or through an USB port. Additionally, the handheld analyte testing device 10 consists of a memory 25 which stores the measurement results. The memory 25 may further contain interactive instructions for using and operating the device to be displayed on the screen 27. The instructions may comprise an interactive feature-rich presentation including a multimedia recording providing audio/video instructions for operating the device, or alternatively simple text, displayed on the screen, illustrating step-by-step instructions for operating and using the device. The inclusion of interactive instructions with the device eliminates the need for extensive training for use, allowing for patient self-testing and use by persons other than medical professionals. In an exemplary embodiment, the memory 25 may also contain a reference database for statistical calibration of the device. In another embodiment, the reference database may be accessed from a remote storage device via a wireless or a wired connection. Similarly, data collected from the subject by the analyte testing device 10 may be recorded in the database for future reference.

The analyte testing device 10 can be a standalone system or can operate in conjunction with a mobile or stationary device to facilitate display or storage of data, and to signal healthcare personnel when therapeutic action is needed, if the device is used for continuous monitoring of a diagnostic parameter associated with a disease state. Mobile devices can include, but are not limited to, handheld devices and wireless devices distant from, and in communication with, the analyte testing device 10. Stationary devices can include, but are not limited to, desktop computers, printers and other peripherals that display or store the results of the test. In an exemplary embodiment, the analyte testing device 10 stores each patient file, which includes a summary of the session and test results, on a removable memory card 21, such as compact flash (CF) card. The user can then use the memory card 21 to transfer patient information and procedural data to a computer, or to produce a printout of the data and session summary. In another embodiment, results from the processor 23 are transferred directly to an external mobile or stationary device to facilitate display or storage of data. For example, the results from the processor 23 may be displayed or stored on a PC 29 using a PC interface, such as an USB port, IRDA port, BLUETOOTH® or other wireless link. In yet another embodiment, the results can be transmitted wirelessly or via a cable to a printer 31 that prints the results to be used by attending medical personnel. Further, the analyte testing device 10 can transmit data to another mobile or stationary device to facilitate more complex data processing or analysis. For example, the device, operating in conjunction with PC 29, can send data to be further processed by the computer.

Although the Optical Bridge™ method and the analyte testing device 10 are described here with a focus towards measuring the concentration of glucose in blood, the method and device presented in this disclosure may also be employed to detect the concentration of other analytes, such as urea, cholesterol, nicotine, drugs, etc., in blood or other fluids. Additionally, the fiberless transflectance probe 20 and its method of use may be utilized in any optical detection system operating in the infrared, visible, or ultraviolet wavelength range.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. An apparatus for noninvasively interrogating a target region for measuring an amount of a target analyte, comprising:
a source for generating a combined beam of electromagnetic radiation including at least two repetitive periods of radiation having different wavelengths, at least two of the wavelengths having different absorption coefficients for the target analyte;
a detector arranged to detect a portion of the radiation backscattered by the target region, the detector generating an output signal proportional to the detected intensity of the combined beam at each of the two repetitive periods of radiation; and
a fiberless transflectance probe for directing the beam of electromagnetic radiation to the target region and conducting the backscattered radiation to the detector;
wherein the fiberless transflectance probe comprises a tapered tubular housing with an inner reflective surface, a cylindrical optical rod with an outer reflective surface and a detection window through which the beam of electromagnetic radiation is transmitted to the target region.

2. The apparatus of claim 1, wherein the target region comprises a fluid.

3. The apparatus of claim 2, further comprising means for compressing and decompressing the target region to control the amount of fluid within the target region.

4. The apparatus of claim 3, wherein the means for compressing and decompressing the target region is a controllable mechanical device.

5. The apparatus of claim 2, further comprising means for obtaining an estimate of the amount of fluid within the sample matrix during a measurement.

6. The apparatus of claim 5, wherein the means for obtaining an estimate of the amount of fluid comprises a source for directing radiation at the target region, the radiation having a wavelength that is preferentially absorbed by a component of the fluid.

7. The apparatus of claim 6, wherein the radiation is green light.

8. The apparatus of claim 2, further comprising means for measuring a phase of pulsation of the fluid within the target region.

9. The apparatus of claim 8, wherein the means for measuring a phase of pulsation of the fluid within the target region comprises a source for directing radiation at the target region, the radiation having a wavelength that is preferentially absorbed by a component of the fluid.

10. The apparatus of claim 1, wherein the apparatus is a handheld unit comprising an on-board processor for calculating the concentration of the target analyte.

11. The apparatus of claim 10, further comprising a graphic display screen.

12. The apparatus of claim 10, further comprising a rechargeable battery.

13. The apparatus of claim 10, further comprising a memory for storing user instructions and measurement results.

14. The apparatus of claim 13, wherein the memory comprises a reference database.

15. The apparatus of claim 10, wherein the handheld unit can communicate with an external device using a wireless communication link.

16. The apparatus of claim 1, wherein the inner surface of the tubular housing is faceted to spread the radiation beam evenly on the target region.

17. The apparatus of claim 1, wherein the cylindrical optical rod is positioned perpendicularly in the center of the detection window.

18. The apparatus of claim 1, wherein the tubular housing is positioned around the cylindrical optical rod.

19. The apparatus of claim 2, wherein the fluid is blood and the target analyte measured is glucose.

20. A transflectance probe for measuring a property of a sample, comprising:
a detection window through which the sample is irradiated;
an optical rod with an outer reflective surface positioned perpendicular to the detection window;
a tapered tubular housing with an inner reflective surface positioned around the optical rod;
at least one light source for irradiating the sample; and
a detector positioned at the proximal end of the optical rod for detecting the light backscattered by the sample.

21. The transflectance probe of claim 20, wherein the cylindrical optical rod, the tubular housing and the detection window are comprised of quartz.

22. The transflectance probe of claim 20, wherein the tubular housing and the detection window are comprised of a thermoplastic polymer.

23. The transflectance probe of claim 22, wherein the tubular housing and the detection window are injection molded.

24. The transflectance probe of claim 20, wherein the outer surface of the optical rod is coated with a reflective coating.

25. The transflectance probe of claim 20, wherein the inner surface of the tubular housing is coated with a reflective coating.

26. The transflectance probe of claim 20, wherein the inner surface of the tubular housing is faceted.

27. The transflectance probe of claim 26, wherein the facets are in the form of convex cylinders.

28. The transflectance probe of claim 27, wherein the number of facets correspond to the number of light sources.

29. The transflectance probe of claim 20, wherein light from the at least one light source is transmitted to the sample by reflecting on the outer surface of the optical rod and the inner surface of the tubular housing.

30. The transflectance probe of claim 29, wherein the light backscattered by the sample is conducted to the detector through the optical rod.

31. The transflectance probe of claim 20, wherein the at least one light source comprises a laser diode.

32. The transflectance probe of claim 31, wherein the laser diode is mounted on a heat sink at the proximal end of the tubular housing.

33. A method of noninvasively interrogating a target region for measuring an amount of a target analyte, comprising the steps of:
providing a fiberless transflectance probe comprising a tapered tubular housing with an inner reflective surface, a detection window and an optical rod with an outer reflective surface positioned perpendicular to the detection window;
providing at least two light sources operating at two different wavelengths for generating a radiation beam consisting of at least two time multiplexed components;
transmitting the radiation beam to the target region by reflecting on the inner surface of the tubular housing and the outer surface of the optical rod;
conducting a backscattered beam from the target region to the detector by reflecting on the inner surface of the optical rod; and
providing a detector that detects the backscattered beam and produces an output signal indicative of the differential absorption of the two wavelengths by the target region.

34. The method of claim 33, wherein the tapered tubular housing is faceted to spread the radiation beam uniformly on the target region.

35. The method of claim 33, wherein the differential absorption signal is used to calculate the concentration of the target analyte.

36. The method of claim 33, wherein the analyte measured is glucose.

37. The method of claim 36, wherein the two wavelengths are about 1380 nm and about 1620 nm.

38. The method of claim 37, wherein the two wavelengths are 1385+/−20 nm and 1630+/−20 nm.
